(19) United States
(12) Patent Application Publication
(10) Pub. No.: US 2025/0114521 A1
(43) Pub. Date: Apr. 10, 2025

(54) INFUSION WITH RAPID FLUID LINE AUTO FLUSH

(71) Applicant: ICU Medical, Inc., San Clemente, CA (US)

(72) Inventors: James Duane Jacobson, Lindenhurst, IL (US); Gerald William Brann, Antiosh, IL (US)

(21) Appl. No.: 18/987,731

(22) Filed: Dec. 19, 2024

Related U.S. Application Data
(63) Continuation of application No. 17/493,692, filed on Oct. 4, 2021.
Application No. 17/493,692 is a continuation of application No. 17/114,359, filed on Dec. 7, 2020, now Pat. No. 11,135,360.

(51) Int. Cl. A61M 5/142; A61M 5/14

(52) U.S. Cl. CPC A61M 5/16827; A61M 5/1407; G16H 20/17; G16H 40/40; G16H 40/67; A61M 2005/1403; A61M 2205/3334; A61M 2205/502; A61M 2205/52; G06K 7/10297; G06K 2007/10504; G06K 19/0723

(57) ABSTRACT

An infusion pump system has a reservoir configured to hold a medicinal fluid, a fluid line in fluid communication with the reservoir and having a terminal fluid delivery end, and an infusion pump. The infusion pump system is configured to determine a fluid line volume corresponding to a volume of the fluid line, wherein the fluid line comprises a fluid line volume of a non-medicinal fluid; draw the medicinal fluid from the reservoir to deliver the medicinal fluid; infuse the medicinal fluid at a flushing rate causing displacement of a volume of the non-medicinal fluid remaining in the fluid line and infusion of the non-medicinal fluid out of the terminal fluid delivery end at the flushing rate; determine that an infused volume of the medicinal fluid equals or exceeds the fluid line volume; and reduce infusion to an infusion rate lower than the flushing rate.

### CROSS-REFERENCE TO RELATED APPLICATIONS

The present application is a continuation of U.S. Ser. No. 17/493,692, filed Oct. 4, 2021, which is a continuation of U.S. Ser. No. 17/114,359, filed Dec. 7, 2020, now U.S. Pat. No. 11,135,360, which are incorporated by reference herein and made part of this specification. The present application is also related to U.S. application Ser. No. 16/301,379, filed Nov. 13, 2018, which is the national stage of International Application No. PCT/US2017/032017, filed May 10, 2017, which claims the benefit of priority from U.S. Provisional No. 62/336,191, filed May 13, 2016, all of which are incorporated by reference in their entireties and made part of this specification.

### BACKGROUND

### Field

The present invention relates to medical devices and infusion pump systems.

Infusion pumps are medical devices that deliver fluids, including nutrients and medications such as antibiotics, chemotherapy drugs, and pain relievers, in controlled amounts. Many types of pumps, including large volume, patient-controlled analgesia (PCA), elastomeric, syringe, enteral, and insulin pumps, are used worldwide in healthcare facilities, such as hospitals, and in the home. Clinicians and patients rely on pumps for safe and accurate administration of fluids and medications.

It is often desirable to provide more than one therapeutic fluid to the patient from the same infusion pump. Two fluid reservoirs with different therapeutic fluids are connected to the infusion pump and then delivered through a common line having a terminal fluid delivery end. The terminal fluid delivery end is attached to the patient. The first therapeutic fluid and second therapeutic fluid may be administered concurrently or one at a time by controlling the fluid flow path to draw fluid from both reservoirs or from only one reservoir.

When switching from single to concurrent fluid delivery, the therapeutic fluid remaining in the common line may lead to complexity in controlling delivery volumes or flow rates when switching between fluid sources. For example, the remaining therapeutic fluid must be cleared from the common line before the next therapeutic fluid begins administration (entering the patient's body), which delays the next therapeutic fluid from reaching the patient. In addition, when the therapeutic fluids are administered concurrently, the first therapeutic fluid remaining in the common line will be administered at the combined rate of the first therapeutic fluid infusion rate plus the second therapeutic fluid infusion rate, e.g., the remaining first therapeutic fluid will be administered at the combined rate determined from the rates specified for the first and second therapeutic fluids. This can result in the patient receiving more or less than the optimum therapy with respect to the first therapeutic fluid. Furthermore, the remaining therapeutic fluid may not be correctly accounted for, potentially creating delays in the values indicated at the infusion pump, versus therapeutic fluid received by the patient. Finally, a single medication delivered at a combined rate may actually result in the single medication being infused at a rate that can exceed an upper soft or hard limit specified for such medication until the medication in the common line is displaced by an intended second fluid (in the case of a piggyback infusion) or a mixture of first and second fluids (in the case of a concurrent delivery). While the pump data will be correct in terms of infusion rates over given times, the actual fluid delivery to the terminal fluid delivery end at the patient may not be correctly captured in pump and system data.

In addition, while some infusion therapies specify a particular volume of fluid to infuse to a patient, in some therapies it is preferred to deliver 100% of the volume of fluid contained within a particular fluid reservoir, such that the fluid is delivered until the reservoir is emptied. However, with many infusion pump systems, due to variable fluid volume contained in the reservoir and typical pump delivery accuracy tolerances and system dependencies, it is only possible to achieve 100% fluid delivery by over-programming the pump, or by entering pump programming parameters that do not accurately reflect the volume and duration of fluid actually administered to the patient.

It would be desirable to have infusion pump systems and methods with common line auto flush that would overcome the above disadvantages.

### SUMMARY

In one embodiment, a control system is provided to control operation of an infusion pump of an infusion pump system. The infusion pump system includes a first reservoir configured to hold a first fluid, a second reservoir configured to hold a second fluid, a junction in fluid communication with the first reservoir and the second reservoir, a common line in fluid communication with the junction and having a terminal fluid delivery end, and the infusion pump, wherein the infusion pump is operable to drive fluid through the common line toward the terminal fluid delivery end. The control system includes: one or more hardware processors; and a memory storing executable instructions that when executed by the one or more hardware processors, configure the infusion pump to: receive instructions to deliver the first fluid at a first rate, subsequently concurrently deliver a mixture of the first fluid and the second fluid, and concurrently deliver the first fluid at the first rate and the second fluid at a second rate; infuse the first fluid at the first rate along a first flow path, the first flow path including the common line; determine a common line volume corresponding to a volume of the common line; draw the first fluid from the first reservoir the second fluid from the second reservoir to deliver the mixture of the first fluid and the second fluid; infuse the mixture of the first fluid and the second fluid at a flushing rate along a second flow path, the second flow path including the common line; determine that an infused volume of the mixture of the first fluid and the second fluid equals or exceeds the common line volume; and change the infusion rate of the mixture of the first fluid and the second fluid from the flushing rate to a combined rate, wherein the combined rate is the sum of the first rate and the second rate, and continue to infuse the mixture of the first fluid and the second fluid along the second flow path at the combined rate.

The control system may also include a mixing chamber in fluid communication with the first reservoir, the second reservoir, and the common line. The executable instructions may further configure the infusion pump to determine the flushing rate based upon whether the first fluid is a medicinal fluid, determine the flushing rate as the first rate when the first fluid is a medicinal fluid, or determine the flushing rate as the first rate increased by a flushing rate factor when the first fluid is not a medicinal fluid.

The instructions may further configure the infusion pump to receive the common line volume from a user input, retrieve the common line volume from the memory, or retrieve the common line volume over a network. The common line volume may be predetermined. The instructions may further configure the infusion pump to determine the common volume based on the first fluid. The first rate may be different than the second rate.

The instructions may further configure the infusion pump to receive the instructions for the delivery from an input via a user interface. The executable instructions further configure the infusion pump to: determine that an infusion of the second fluid has completed; draw the first fluid from the first reservoir without drawing the second fluid from the second reservoir; infuse the first fluid at the combined rate; determine that a volume of the first fluid infused at the combined rate equals or exceeds the common line volume; and change the infusion rate of the first fluid from the combined rate to the first rate.

The executable instructions may configure the infusion pump to determine that an infusion of the second fluid has completed by comparing a volume of fluid infused to a programmed volume to infuse, determine that an infusion of the second fluid has completed by receiving an instruction to stop infusing the second fluid, or determine that an infusion of the second fluid has completed by determining that the second reservoir has been depleted of second fluid.

The executable instructions may further configure the infusion pump to: determine that an infusion of the first fluid has completed; draw the second fluid from the second reservoir without drawing the first fluid from the first reservoir; infuse the second fluid at the combined rate; determine that a volume of the second fluid infused at the combined rate equals or exceeds the common line volume; and change the infusion rate of the second fluid from the combined rate to the second rate.

The executable instructions may configure the infusion pump to determine that an infusion of the first fluid has completed by comparing a volume of fluid infused to a programmed volume to infuse, determine that an infusion of the first fluid has completed by receiving an instruction to stop infusing the first fluid, or determine that an infusion of the first fluid has completed by determining that the first reservoir has been depleted of first fluid.

In another embodiment, a method for controlling operation of an infusion pump of an infusion pump system is provided. The infusion pump system includes a first reservoir configured to hold a first fluid, a second reservoir configured to hold a second fluid, a junction in fluid communication with the first reservoir and the second reservoir, a common line in fluid communication with the junction and having a terminal fluid delivery end, and the infusion pump, wherein the infusion pump is operable to drive fluid through the common line toward the terminal fluid delivery end. The method includes: drawing the first fluid from the first reservoir and the second fluid from the second reservoir to form a mixture of the first fluid and the second fluid; infusing the mixture of the first fluid and the second fluid at a combined rate, wherein the combined rate is a sum of a first infusion rate associated with the first fluid and a second infusion rate associated with the second fluid; determining a common line volume corresponding to a volume of the common line; determining that the second reservoir is depleted; drawing the first fluid from the first reservoir without drawing the second fluid from the second reservoir; driving the first fluid at the combined rate along a flow path including the common line; determining that a driven volume of the first fluid equals or exceeds the common line volume; and changing the infusion rate of the first fluid from the combined rate to the first rate, and continuing to infuse the first fluid along the flow path at the first rate.

The infusion pump may also include a mixing chamber in fluid communication with the first reservoir, the second reservoir, and the common line. Determining the common line volume may include receiving the common line volume from a user input, retrieving the common line volume from a memory, or retrieving the common line volume over a network. The common line volume may be predetermined.

Determining the common line volume may include determining the common line volume based on the first fluid. The first rate may be different than the second rate. Driving the first fluid at the combined rate may include driving the first fluid at a rate that exceeds a drug library rate limit associated with the first fluid. Determining that the second reservoir is depleted may include receiving a sensor signal that air is present in the junction or in a line coupling the junction to the second reservoir.

The method may further include pumping the first fluid from the first reservoir towards the second reservoir in response to receiving the sensor signal that air is present in the junction or in the line coupling the junction to the second reservoir.

In yet another embodiment, a control system for controlling operation of an infusion pump of an infusion pump system is provided. The infusion pump system includes a first reservoir configured to hold a first fluid, a second reservoir configured to hold a second fluid, a junction in fluid communication with the first reservoir and the second reservoir, a common line in fluid communication with the junction and having a terminal fluid delivery end, and the infusion pump, wherein the infusion pump is operable to drive fluid through the common line toward the terminal fluid delivery end. The control system includes: one or more hardware processors; and a memory storing executable instructions that when executed by the one or more hardware processors, configure the infusion pump to: draw the first fluid from the first reservoir and the second fluid from the second reservoir to form a mixture of the first fluid and the second fluid; infuse the mixture of the first fluid and the second fluid at a combined rate, wherein the combined rate is a sum of a first infusion rate associated with the first fluid and a second infusion rate associated with the second fluid; determine a common line volume corresponding to a volume of the common line; draw the first fluid from the first reservoir without drawing the second fluid from the second reservoir; drive the first fluid at the combined rate along a flow path including the common line; determine that a driven volume of the first fluid equals or exceeds the common line volume; and change the infusion rate of the first fluid from the combined rate to the first rate, and continue to infuse the first fluid along the flow path at the first rate.

The infusion pump may also include a mixing chamber in fluid communication with the first reservoir, the second reservoir, and the common line.

The executable instructions may also configure the infusion pump to determine the common line volume by receiving the common line volume from a user input, determine the common line volume by retrieving the common line volume from a memory, or determine the common line volume by retrieving the common line volume over a network. The common line volume may be predetermined.

The executable instructions may also configure the infusion pump to determine the common line volume by determining the common line volume based on the first fluid. The first rate may be different than the second rate.

The executable instructions may configure the infusion pump to drive the first fluid at the combined rate by driving the first fluid at a rate that exceeds a drug library rate limit associated with the first fluid. The executable instructions may configure the infusion pump to determine that the second reservoir is depleted by receiving a sensor signal that air is present in the junction or in a line coupling the junction to the second reservoir. The executable instructions may further configure the infusion pump to pump the first fluid from the first reservoir towards the second reservoir in response to receiving the sensor signal that air is present in the junction or in the line coupling the junction to the second reservoir.

In yet another embodiment, a method for controlling operation of an infusion pump of an infusion pump system is provided. The infusion pump system includes a first reservoir configured to hold a first fluid, a second reservoir configured to hold a second fluid, a junction in fluid communication with the first reservoir and the second reservoir, a common line in fluid communication with the junction and having a terminal fluid delivery end, and the infusion pump, wherein the infusion pump is operable to drive fluid through the common line toward the terminal fluid delivery end. The method includes: receiving instructions to deliver the first fluid at a first rate, subsequently concurrently deliver a mixture of the first fluid and the second fluid, and concurrently deliver the first fluid at the first rate and the second fluid at a second rate; infusing the first fluid at the first rate along a first flow path, the first flow path including the common line; determining a common line volume corresponding to a volume of the common line; drawing the first fluid from the first reservoir the second fluid from the second reservoir to deliver the mixture of the first fluid and the second fluid; infusing the mixture of the first fluid and the second fluid at a flushing rate along a second flow path, the second flow path including the common line; determining that an infused volume of the mixture of the first fluid and the second fluid equals or exceeds the common line volume; and changing the infusion rate of the mixture of the first fluid and the second fluid from the flushing rate to a combined rate, wherein the combined rate is the sum of the first rate and the second rate, and continue to infuse the mixture of the first fluid and the second fluid along the second flow path at the combined rate.

The infusion pump system may also include a mixing chamber in fluid communication with the first reservoir, the second reservoir, and the common line. The method may also include determining the flushing rate based upon whether the first fluid is a medicinal fluid, determining the flushing rate as the first rate when the first fluid is a medicinal fluid, or determining the flushing rate as the first rate increased by a flushing rate factor when the first fluid is not a medicinal fluid.

The method may also include receiving the common line volume from a user input, retrieving the common line volume from the memory, or retrieving the common line volume over a network. The common line volume may be predetermined.

The method may also include determining the common volume based on the first fluid. The first rate may be different than the second rate. Infusing the mixture of the first fluid and the second fluid at the flushing rate may include one or more of delivering the first fluid at a first fluid flush rate that exceeds a drug library rate limit associated with the first fluid or delivering the second fluid at a second fluid flush rate that exceeds a drug library rate limit associated with the second fluid.

The method may also include: determining that an infusion of the second fluid has completed; drawing the first fluid from the first reservoir without drawing the second fluid from the second reservoir; infusing the first fluid at the combined rate; determining that a volume of the first fluid infused at the combined rate equals or exceeds the common line volume; and changing the infusion rate of the first fluid from the combined rate to the first rate.

The method may also include determining that an infusion of the second fluid has completed by comparing a volume of fluid infused to a programmed volume to infuse, determining that an infusion of the second fluid has completed by receiving an instruction to stop infusing the second fluid, or determining that an infusion of the second fluid has completed by determining that the second reservoir has been depleted of second fluid. Infusing the first fluid at the combined rate may include infusing the first fluid at a rate that exceeds a drug library rate limit associated with the first fluid.

The method may also include: determining that an infusion of the first fluid has completed; drawing the second fluid from the second reservoir without drawing the first fluid from the first reservoir; infusing the second fluid at the combined rate; determining that a volume of the second fluid infused at the combined rate equals or exceeds the common line volume; and changing the infusion rate of the second fluid from the combined rate to the second rate.

The method may also include determining that an infusion of the first fluid has completed by comparing a volume of fluid infused to a programmed volume to infuse, determining that an infusion of the first fluid has completed by receiving an instruction to stop infusing the first fluid, or determining that an infusion of the first fluid has completed by determining that the first reservoir has been depleted of first fluid. Infusing the second fluid at the combined rate may include infusing the second fluid at a rate that exceeds a drug library rate limit associated with the second fluid.

The foregoing and other features and advantages of the invention will become further apparent from the following detailed description of the presently preferred embodiments, read in conjunction with the accompanying drawings. The detailed description and drawings are merely illustrative of the invention rather than limiting. The scope of the invention is defined by the appended claims and equivalents thereof.

In certain embodiments, a control system can control operation of an infusion pump system. The infusion pump system can include a first reservoir that can hold a first fluid, a second reservoir configured to hold a second fluid, a junction in fluid communication with the first reservoir and the second reservoir, a common line in fluid communication with the junction and having a terminal fluid delivery end, and an infusion pump operable to drive fluid through the common line toward the terminal fluid delivery end. The control system can control whether fluids from the reservoirs are drawn individually or concurrently (e.g., simultaneously or in an alternating manner). For example, the control system can include a flow control mechanism to manipulate a flow path at the junction to draw fluid from the first reservoir alone, the second reservoir alone, or both first and second reservoirs in an alternating manner.

The first reservoir may be referred to as the primary source and the second reservoir may be referred to as the secondary source. During a primary infusion, fluid is infused from the first, or primary reservoir, into the junction, and through the common line to the terminal end (and into the patient) at a first infusion rate. During a secondary infusion, fluid is infused from the second, or secondary reservoir, into the junction, and through the common line to the terminal end (and into the patient) at a second infusion rate. During a concurrent infusion (sometimes referred to as concurrent delivery), first and second fluids are infused simultaneously to a patient at respective first and second infusion rates. A first volume of the first fluid is drawn from the first reservoir, and a second volume of the second fluid is drawn from the second reservoir. The first and second volumes are proportionate to the first and second infusion rates. Once the first and second fluids have been drawn, the pump drives (e.g., pumps or pushes out) the fluid combination through the common line to the terminal and (and into the patient) at a combined rate.

The combined rate can be equal to one of the first or second infusion rates, or it can be determined from the first and second infusion rates. For example, the combined rate can be determined as the sum as the first and second infusion rates. In some cases, a maximum rate may be established, and if the sum of the programmed first and second rates exceeds the maximum rate, then the combined rate may be set to the maximum rate. Other methods of determining a combined rate using the first and second rates are possible, as well. In addition, if the combined rate equals or exceeds a predetermined maximum combined rate, the first and second rates may be reduced proportionally such that their sum is less than or equals the maximum combined rate. In other embodiments, if the combined rate equals or exceeds a predetermined maximum combined rate, only the first rate is reduced until the sum of the first and second rates is less than or equals the maximum combined rate. For example, only the first rate may be reduced based upon a determination of fluid types of the first and second fluids. If the first fluid is a non-medication and the second fluid is a medication, then in some embodiments, the only the first rate is reduced (or the first rate is reduced by an amount or proportion that is greater than an amount or proportion that the second rate is reduced), such that the sum of the first and second rates is less than or equal to the maximum combined rate. In such embodiments, the user would be presented with a suggested first and second rate for approval or confirmation via a user interface before changing and/or initiating an infusion according to such adjusted first and/or second rates.

### BRIEF DESCRIPTION OF THE DRAWINGS

FIGS. 1A and 1B are block diagrams of infusion pump systems with concurrent fluid delivery and common line auto flush in accordance with the present invention.

FIG. 2 is a block diagram of an infusion pump with concurrent fluid delivery and common line auto flush in accordance with the present invention.

FIG. 3 is a schematic diagram of an infusion pump with concurrent fluid delivery and common line auto flush in accordance with the present invention.

FIGS. 4A and 4B are graphs of fluid volume delivered at the terminal fluid delivery end of the common line versus time for a method of use for an infusion pump with concurrent fluid delivery and common line auto flush in accordance with the present invention.

FIG. 5A is a flowchart of a method of concurrent fluid delivery and common line auto flush in accordance with the present invention that may be performed by the infusion pumps of FIGS. 1-3.

FIG. 5B is a flowchart of a method of providing a secondary infusion until the secondary reservoir is depleted in accordance with the present invention that may be performed by the infusion pumps of FIGS. 1-3.

FIG. 5C is a flowchart of a method of providing sequential infusions, where a primary infusion is provided until the primary reservoir is depleted in accordance with the present invention that may be performed by the infusion pumps of FIGS. 1-3.

FIG. 6 is a flowchart of a method of determining fluid drive start times to cause infusions to reach a patient at desired infusion start times in accordance with the present invention that may be performed by the infusion pumps of FIGS. 1-3.

FIGS. 7A-7E are schematic diagrams of use for an infusion pump system with concurrent fluid delivery and common line auto flush in accordance with the present invention.

Like elements share like reference numbers throughout the various figures.

### DETAILED DESCRIPTION

Systems and methods that improve an infusion pump system with concurrent delivery and common line auto flush are described herein. An infusion pump can operate in a primary delivery mode and deliver a first fluid from a first reservoir at a first rate, and then switch to a concurrent delivery mode, such as by delivering a combination of the first fluid from the first reservoir and a second fluid from a second reservoir at a combined delivery rate. The pump may switch from a concurrent delivery mode to a primary delivery mode (or to a secondary delivery mode where a second fluid is delivered from a second reservoir at a second rate), as well.

As discussed above, first fluid will remain in the common line at the time the delivery mode is switched from primary delivery mode to concurrent delivery mode. Therefore, if the first and second fluids are delivered at the combined delivery rate as soon as the concurrent delivery mode begins, the first fluid remaining in the common line will be delivered into the patient at the incorrect (i.e., the combined) rate. Furthermore, delivering fluids at rates other than the desired rates may result in inaccurate therapy, which can be dangerous to the patient. The systems and methods described herein improve delivery and accurately account for the fluid remaining in the volume of the common line. Fluid as used herein can be any fluid suitable to be administered to a patient by infusion, including saline fluid, fluid including a drug or other therapeutic agent, or the like.

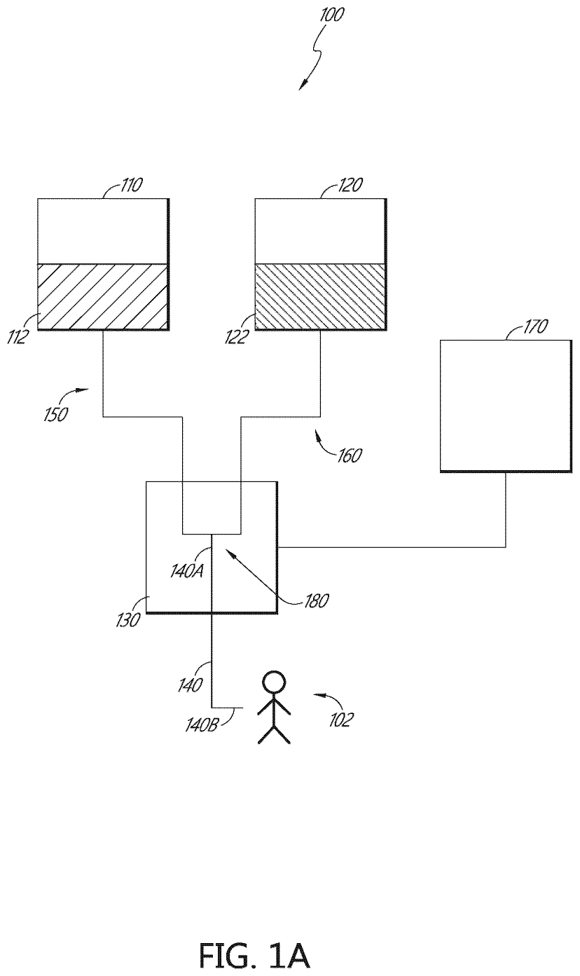

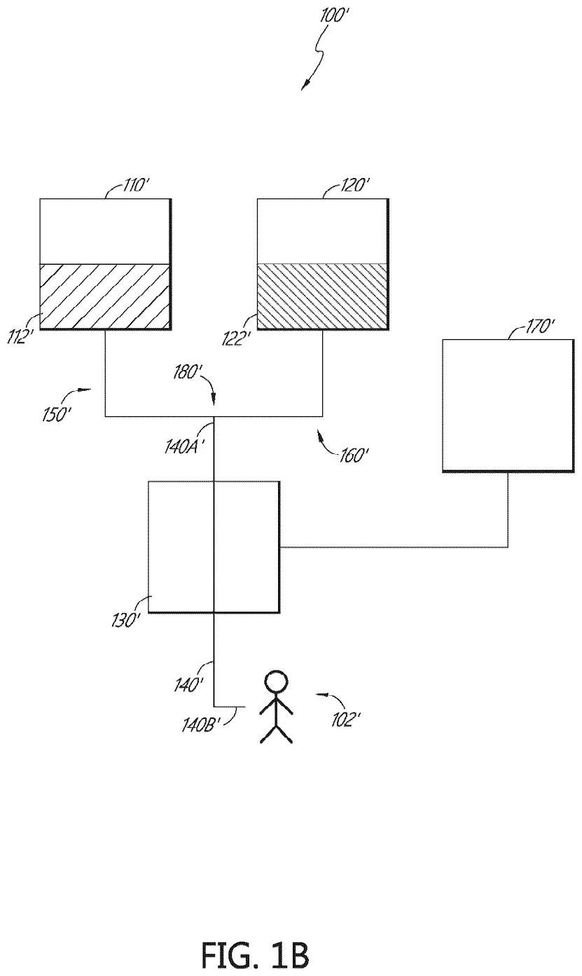

FIGS. 1A & 1B are block diagrams for embodiments of infusion pump systems with concurrent delivery and a common line. The infusion pump system illustrated in FIG. 1A includes a junction in fluid communication with the first reservoir and the second reservoir. An optional mixing chamber is located at the junction, or between the junction and the common line. The junction and/or mixing chamber is located internal to the infusion pump. In the embodiment of the infusion pump system illustrated in FIG. 1B a junction in fluid communication with the first reservoir and the second reservoir is located external to the infusion pump. An optional mixing chamber is located at the junction, or between the junction and the common line. The location of the junction, in part, determines the length and internal volume of the common line between the junction and the terminal fluid delivery end. The internal cross-sectional shape, which is usually substantially circular, and the diameter and length of the common line determine its internal volume. Other shapes can be used without detracting from the scope of the disclosure.

The infusion pump system 100 of FIG. 1A includes a junction 180 internal to the infusion pump 130 and an optional mixing chamber (not shown) at the junction or between the junction 180 and a common line 140. The infusion pump system 100 includes a first reservoir 110 that contains a first fluid 112; a second reservoir 120 that contains a second fluid 122; a junction 180 in fluid communication with the first reservoir 110 and the second reservoir 120; an optional mixing chamber (not shown); a common line 140 in fluid communication with the mixing chamber and/or the junction 180 at one end 140A and having a terminal fluid delivery end 140B for connection to the patient 102, and an infusion pump 130 operable to drive fluid through the common line 140.

### Primary Infusion Mode

The infusion pump 130 is operable to operate in a primary infusion mode during which the infusion pump infuses the first fluid 112 at a first rate along a first flow path 150 that includes the first reservoir 110, the junction 180, the optional mixing chamber, and the common line 140. The infusion pump 130 is further operable to determine a common line flush volume value corresponding to the internal volume of the common line 140. The infusion pump 130 may determine the common line flush volume by receiving the value from an operator, receiving it over a network (e.g., from a drug library or other database), retrieving it from a memory of the infusion pump, or any other method described herein.

### Primary to Concurrent Infusion Mode With Auto Flush

The infusion pump 130 is further configured to change to a concurrent infusion mode by drawing a second fluid 122 from a second reservoir 120 along a second flow path 160 into the junction 180 and/or mixing chamber and mixing it with the first fluid 112, drawn from the first reservoir 110 via the first flow path 150. The second flow path 160 includes the second reservoir 120, the junction 180, and the optional mixing chamber. The infusion pump is configured to initially infuse the mixture at the first rate until the volume of the first fluid is flushed out of the common line 140. The infusion pump 130 is configured to monitor volume of the mixture of first and second fluids 112, 122 driven at the first rate and subsequently pump the mixture of first and second fluids 112, 122 at the programmed combined rate when the monitored volume of the mixture is equal to or greater than the common line flush volume value. In this case, the delivery rates of fluid 1 and fluid 2 would be reduced (scaled down) from programmed rates during displacement of the common line volume, and the pump system may allow an override of one or both lower rate limits, or other associated limits, defined respectively for each of fluid 1 and fluid 2, during this phase of delivery.

Alternatively, for example in a scenario where the first fluid is a not a medication (e.g., saline) and it is desired to initiate delivery of the second fluid (that is a medication) rapidly, the infusion pump could be configured to initially infuse the mixture at a more rapid rate to quickly displace the relatively inert common line volume. In this case, the initial combined rate could be increased (scaled up) to the programmed first rate plus the programmed second rate until the monitored volume of the mixture is equal to the common line flush volume. In this case, the delivery rates of fluid 1 and/or fluid 2 may be increased above upper rate limits defined for those respective fluids and the pump system may allow override of those limits during this phase of delivery. Further, the resulting scaled combined rate will be applied to the common line fluid 1, whose upper rate limit may limit or define allowable increased combined rates during the common line displacement phase. In this case, drug library defined limits would be considered and applied by the pump system at the point of infusion to the patient as well as per pump programming activity.

### Concurrent to Primary Infusion Mode With Auto Flush

The infusion pump 130 is further configured to change from concurrent delivery to a primary infusion mode by refraining from drawing the second fluid 122 from the second reservoir 120, and by infusing only the first fluid 112 from the first reservoir 110 along the first flow path 150. When the infusion pump switches to primary infusion mode, the infusion pump is configured to initially drive the first fluid 112 at the combined rate until the volume of the mixture of first and second fluids 112, 122 is flushed out of the common line 140. The infusion pump 130 is configured to monitor volume of the first fluid 112 driven at the combined rate and subsequently pump the first fluid 112 at the first rate when the monitored first fluid volume is equal to or greater than the common line flush volume value. In one example, the infusion pump 130 can be a fluid displacement pump employing a cassette, such as the Plum 360™ infusion pump available from ICU Medical, Inc. of San Clemente, CA. Those skilled in the art will appreciate that the infusion pump 130 can be any type of pump operable to drive fluid from two reservoirs through a common line 140. In this case, driving of the first fluid at the combined rate during common line displacement may require that the pump system allows override of drug library-defined upper rate limits for the first fluid.

### Concurrent to Secondary Infusion Mode With Auto Flush

In another embodiment, the infusion pump 130 is further configured to change from a concurrent delivery to a secondary infusion mode by refraining from drawing the first fluid 112 from the first reservoir 110, and by infusing only the second fluid 122 from the second reservoir 120 along the second flow path 160. For example, if the first fluid 112 infusion is completed or is stopped, the infusion pump 130 may automatically switch to a secondary infusion mode. In such case, the infusion pump 130 will stop drawing first fluid 112 from the first reservoir 110, and it will continue to pump at the combined rate until the common line is cleared of the fluid mixture. In this case, the pump system may need to allow an override of upper rate limits for the second fluid while it is pumped at the combined rate during common line displacement. The infusion pump 130 is configured to monitor volume of the second fluid 122 driven at the combined rate and subsequently pump the second fluid 122 at the second rate when the monitored second fluid volume is equal to or greater than the common line flush volume value.

In one embodiment, the infusion pump 130 can be operably connected to a medication management unit (MMU) 170 or a server over a hospital network and/or the Internet, to receive a drug library (or other database), which may specify an appropriate common line flush volume value. For example, the drug library (or other database) may include information regarding the volume of various tubing assemblies, each tubing assembly including a common line. The infusion pump (or server) may be configured to determine a tubing assembly identifier associated with a tubing assembly that is attached to the infusion pump and the patient 102. The infusion pump may determine the tubing assembly identifier by receiving it from a server over the hospital network and/or the Internet, by receiving it via manual data entry by an operator, and/or by reading the tubing assembly identifier from the tubing assembly (or by other methods). For example, a tag, such as an RFID tag, an NFC tag or other wireless tag, may include the tubing assembly identifier. A tag reader incorporated into or in communication (directly or indirectly) with the infusion pump, may read the tag to determine the tubing assembly identifier. The common line flush volume value may be determined using the tubing assembly identifier and the drug library (or database).

In one embodiment, the infusion pump 130 can be further operable to increment a displayed value of first fluid volume by the monitored volume when the mixture of first and second fluids 112, 122 are driven at the first rate. The infusion pump 130 can be further operable to increment a displayed value of first and second fluid volumes when the first fluid 112 is driven at the combined rate. In one embodiment, the infusion pump 130 is operable to monitor the volume of infused first fluid 112 and switch to a concurrent infusion mode when the volume of the infused first fluid is equal to a Volume To Be Infused (VTBI) for the first fluid or when the volume of the infused first fluid is equal to the VTBI for the first fluid minus the volume of the common line. In one embodiment, the infusion pump 130 is operable to monitor the volume of infused second fluid 112 during a concurrent infusion mode and switch to a primary infusion mode when the volume of the infused second fluid is equal to a Volume To Be Infused (VTBI) for the second fluid or when the volume of the infused second fluid is equal to the VTBI for the second fluid minus the volume of the common line.

The infusion pump 130 can be operable to receive the common line flush volume value for the common line 140 automatically from the drug library stored in a memory locally in the infusion pump system 100 or remotely on a server. In one example, the drug library associates the common flush volume value with a particular therapeutic agent. In some cases, the drug library may include an indication (e.g., a flag, value, etc.) that a particular fluid is a rate dependent medicinal fluid whose action is rate dependent. The infusion pump 130 may be configured to infuse such fluids (whether alone or concurrently with a second fluid) at the infusion rate specified for such fluids. In another example, the drug library associates the common flush volume value with a particular clinical care area (CCA), such as general care, an intensive care unit (ICU), a neonatal ICU, or the like. In yet another example, the drug library associates the common flush volume value with a particular consumable infusion set, which provides the common line volume. The drug library can include upper and lower dosing limits with hard and soft limits for a number of therapeutic agents. In another embodiment, the infusion pump 130 can be operable to receive the common line flush volume value for the common line 140 from a caregiver via an input on a user interface of the infusion pump.

The common line 140 as illustrated includes the line between the junction 180 and the terminal fluid delivery end 140B that is generally connectable to the patient 102 and includes any fluid path common to the first flow path 150 and the second flow path 160. Thus, the common line 140 can include flow paths within the infusion pump 130 (including the associated consumable infusion set, when applicable) common to the first flow path 150 and the second flow path 160, and is not limited to tubing external to the infusion pump 130. The common line 140 is any portion of the infusion pump system 100 through which the first fluid 112 or a combination of the first fluid 112 and the second fluid 122 can alternately flow when switched. In one embodiment, the common line flush volume value is an internal volume of the common line 140. The common line flush volume value can include an associated consumable infusion set volume, extension sets, filters, stopcocks, manifolds, patient access devices, catheters, and the like. In another embodiment, the common line flush volume value is an internal volume of the common line 140 plus an adjustment volume. The adjustment volume can be any volume desired as a safety factor to assure that the common line 140 is free of the first fluid 112 before the second fluid 122 is infused at the second rate.

The infusion pump system 100′ of FIG. 1B has a junction 180′ external to the infusion pump 130′ and an optional mixing chamber (not shown) at the junction 180′ or between the junction 180′ and a common line 140′. The infusion pump system 100′ includes a first reservoir 110′ containing a first fluid 112′; a second reservoir 120′ containing a second fluid 122′; a junction 180′ in fluid communication with the first reservoir 110′ and the second reservoir 120′; an optional mixing chamber (not shown); a common line 140′ in fluid communication with the mixing chamber and/or the junction 180′ and the terminal fluid delivery end 140B′ that is generally connectable to the patient 102′, and an infusion pump 130′ operable to drive fluid through the common line 140′. The infusion pump 130′ is operable to: infuse the first fluid 112′ at a first rate along a first flow path 150′ including the first reservoir 110′, the junction 180′, the optional mixing chamber, and the common line 140′; determine a common line flush volume value for the common line 140′. The infusion pump 130′ may determine the common line flush volume by receiving the value from an operator, receiving it over a network (e.g., from a drug library or other database), retrieving it from a memory of the infusion pump, or any other method described herein.

The infusion pump 130′ is further configured to change to a concurrent infusion mode by drawing a second fluid from a second reservoir 120′ along a second flow path 160′ into the optional mixing chamber and mixing it with the first fluid, drawn from the first reservoir 110′ via the first flow path 150′. The second flow path 160′ includes the second reservoir 120′, the junction 180′, and the optional mixing chamber. The infusion pump is configured to initially infuse the mixture at the first rate until the volume of the first fluid is flushed out of the common line 140′. The infusion pump 130′ is configured to monitor volume of the mixture of first and second fluids 112′, 122′ driven at the first rate and subsequently pump the mixture of first and second fluids 112′, 122′ at a combined rate when the monitored volume is equal to or greater than the common line flush volume value.

The infusion pump 130′ is further configured to change to a primary infusion mode by refraining from drawing the second fluid 122′ from the second reservoir 120′, and by infusing only the first fluid 112′ from the first reservoir 110′ along the first flow path 150′. When the infusion pump 130′ switches to primary infusion mode, the infusion pump 130′ is configured to initially infuse the first fluid 112′ at the combined rate until the volume of the mixture of first and second fluids 112′, 122′ is flushed out of the common line 140′. The infusion pump 130′ is configured to monitor volume of the first fluid 112′ driven at the combined rate and subsequently pump the first fluid 112′ at the first rate when the monitored volume is equal to or greater than a common line flush volume value. The infusion pump 130′ is further configured to determine the common line flush volume value according to any of the methods described herein.

In one embodiment, the junction 180′ can include a two-way valve to manually or automatically switch the infusion pump system 100′ between the first flow path 150′ and the second flow path 160′. In one example, the infusion pump 130′ can be a peristaltic pump. Those skilled in the art will appreciate that the infusion pump 130′ can be any type of pump operable to drive fluid through the common line 140′.

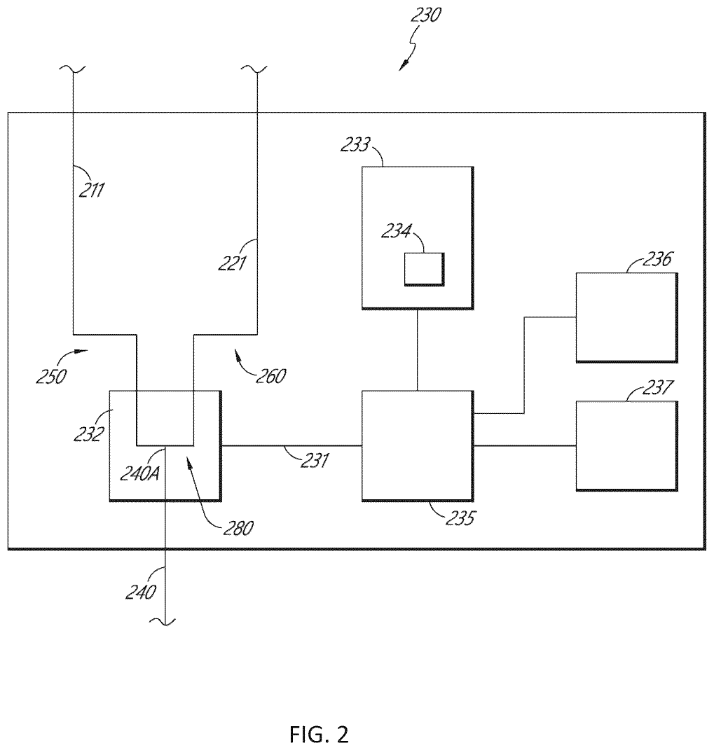

FIG. 2 is a block diagram of an embodiment of an infusion pump with concurrent fluid delivery and common line auto flush. The infusion pump 230 is operably connected to a common line 240 in fluid communication with a junction 280 and/or mixing chamber at one end 240A and having a terminal fluid delivery end 240B (not shown), the junction 280 being in fluid communication with a first reservoir (not shown) containing a first fluid and a second reservoir (not shown) containing a second fluid. In this example, a first reservoir line 211 provides fluid communication between the first reservoir and the junction 280 and a second reservoir line 221 provides fluid communication between the second reservoir and the junction 280.

The infusion pump 230 includes a memory 233 operable to store programming code; a flow controller 235 operably connected to the memory 233; and a fluid driver 232 operably connected to receive a control signal 231 from the flow controller 235, the fluid driver 232 being operable to drive fluid through the common line 240. The flow controller 235 is operable to execute the programming code and provide the control signal 231 to the fluid driver 232 in response to the programming code. The fluid driver 232 is responsive to the control signal 231 to infuse the first fluid at a first rate along a first flow path 211 including the first reservoir, the junction 280, and the common line 240; receive a common line flush volume value associated with the common line 240; switch from infusing only the first fluid via the first flow path 250 to infusing a combination of the first fluid from the first reservoir and a second fluid from the second reservoir; drive the fluid combination at the first rate; monitor volume of the fluid combination driven at the first rate; and drive the fluid combination at a combined rate when the monitored volume is equal to or greater than the common line flush volume value. The fluid driver 232 is also responsive to the control signal 231 to infuse the fluid combination at the combined rate; switch to infusing only the first fluid via the first flow path 250; drive the first fluid at the combined rate; monitor the volume of the first fluid driven at the combined rate; and drive the first fluid at the first rate when the monitored volume is equal to or greater than the common line flush volume value. The combined rate may be retrieved from the memory 233 or determined from a first infusion rate associated with the first fluid and a second infusion rate associated with the second fluid. For example, the combined rate may be determined as the sum of the first and second infusion rates.

In an embodiment, the flow controller 235 monitors the volume based on a time elapsed and a rate of delivery. The flow controller 235 can also monitor volume based on measurements, such as number of turns of a motor or signals from a sensor.

The flow controller 235 can include a hardware processor, microprocessor, or the like responsive to the programming code to generate the control signal 231. The fluid driver 232 can include a metered pump, such as a cartridge pump, peristaltic pump, or the like, operable to drive fluid at a desired rate in response to the control signal 231. In one embodiment, the fluid driver 232 can be further responsive to the control signal 231 to increment a displayed first fluid volume by the monitored volume when the fluid combination is driven at the first rate or when the monitored volume is equal to or greater than an internal volume of the common line 240. The fluid driver 232 can be further responsive to the control signal 231 to increment displayed first and second fluid volumes as the first fluid is driven at the combination rate or when the monitored volume is equal to or greater than the internal volume of the common line 240. The first fluid displayed volume and/or the second fluid displayed volume can be displayed on a user interface 236.

The memory 233 can also be operable to store data and other information, such as a drug library 234 (or other database) including the common flush volume value, which can optionally be associated with a particular therapeutic agent, a particular clinical care area, and/or a particular consumable infusion set. Different therapeutic agents may have different fluid properties and thus it may be advantageous in some embodiments to associate particular common flush volume value with particular therapeutic agents. In one embodiment, the infusion pump 230 can receive the common line flush volume value for the common line 240 automatically from the drug library 234. In another embodiment, the infusion pump 230 can receive the common line flush volume value manually via direct entry of the value on a user interface 236. The manual entry can be accomplished using a manufacturer provided volume value based upon the length and internal diameter of the common line 240 or a list number or other identifier that is used to access an associated volume value from a lookup table in the pump memory 233, drug library, stored in a network location, at a server, or MMU. The possibility for manual typographical errors can be reduced by use of a barcode, radio frequency (RFID), optical, touch memory reader, near field communicator, or the like to input or scan a machine readable identifier on the infusion set, common line, or its package to obtain the volume value, the list number or other identifier associated with the volume value.

The infusion pump 230 can include human and/or machine interfaces as desired for a particular application. A user interface 236 operably connected to the flow controller 235 can provide input from and/or output to a caregiver or other user to the infusion pump 230. Exemplary user interfaces can include display screens, soft keys or fixed keys, touchscreen displays, and the like. An I/O interface 237 operably connected to the flow controller 235 can provide input from and/or output to hardware associated with the infusion pump 230. Exemplary I/O interfaces can include a wired and/or wireless interface to an electronic network, medication management unit (MMU), medication management system (MMS), or the like.

The common line flush volume value can be selected as desired for a particular application. The common line 240 includes the line between the junction 280 and the terminal fluid delivery end 240B, and includes any fluid path common to the first flow path 250 and the second flow path 260 and so can include any portion of the infusion pump 230 (including the associated consumable infusion set) through which the first fluid or the second fluid can alternately flow or flow in a combined manner. In one embodiment, the common line flush volume value is equal to the internal volume of the common line 240, so that the second fluid is infused at the second rate along the second flow path as soon as the first fluid has been cleared from the common line 240. In another embodiment, the common line flush volume value is equal to the internal volume of the common line 240 plus an adjustment volume (to take into account the added/subtracted volume of other connectors or components), so that the second fluid is infused at the second rate along the second flow path after the first fluid has been cleared from the common line 240 plus the adjustment volume of the second fluid has been delivered at the first rate. In another embodiment, the common line flush volume value is equal to the internal volume of the common line modified by a percentage, which could provide a desired overage or underage. The adjustment volume can be used as a safety factor to assure that the common line 240 is free of the first fluid before the second fluid is infused at the second rate.

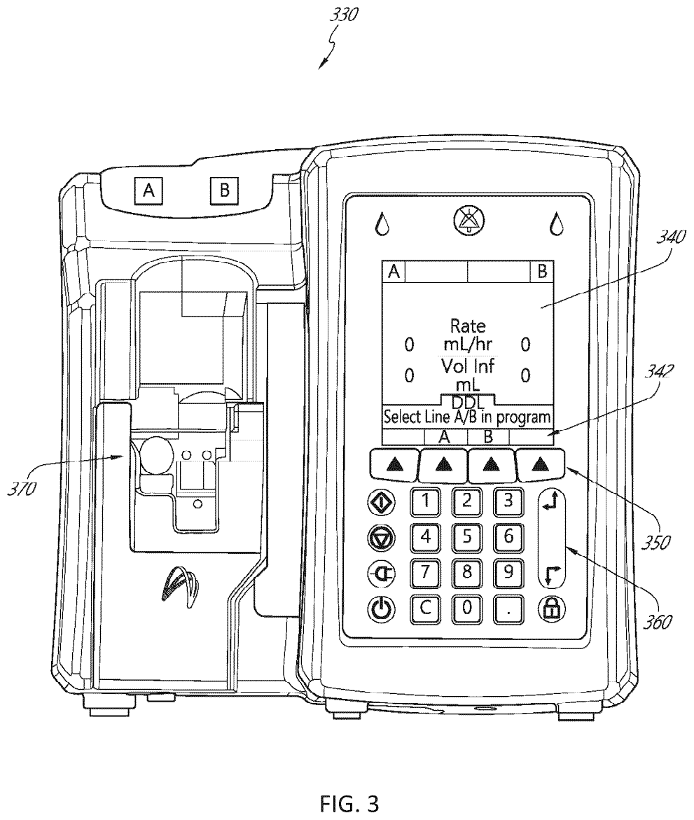

FIG. 3 is a schematic diagram of an infusion pump with common line auto flush in accordance with the present invention. In this example, the infusion pump 330 includes a display 340, soft keys 350, and fixed keys 360 as a user interface. The display 340 provides operational and/or programming information to the user. The soft keys 350 perform different functions depending on the command displayed on an adjacent command portion 342 of the display 340. The fixed keys 360 are labeled with an input or function which functions the same, regardless of whatever is displayed on the display 340. In this example, the infusion pump 330 also includes a pump mechanism 370 operable to communicate with the first reservoir line and the second reservoir line and to move the first fluid or the second fluid to the terminal fluid delivery end of the common line.

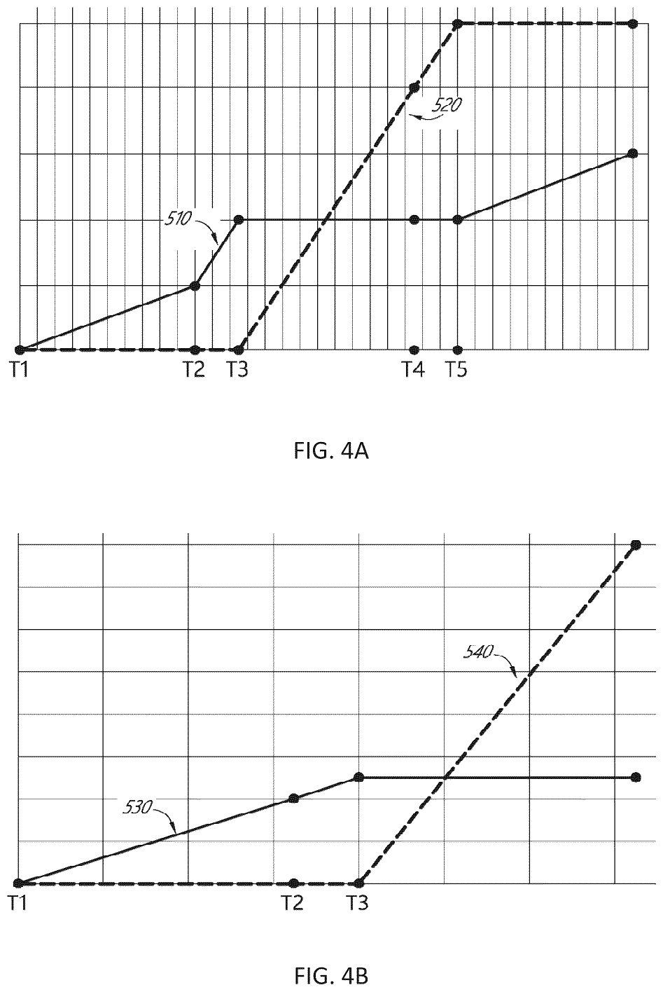

FIGS. 4A & 4B are graphs of fluid volume delivered at the terminal end of the common line or patient versus time for a method of use for an infusion pump with common line auto flush in accordance with the present invention.

Referring to FIG. 4A, graph 510 is the fluid volume delivered at the terminal fluid delivery end of the common line for a first fluid versus time and graph 520 is the fluid volume delivered at the terminal fluid delivery end of the common line for a mixture of the first fluid and a second fluid versus time. From T1 to T2, the first fluid is infused at a first rate along a first flow path including the first reservoir and the second fluid is not infused. From T2 to T3, the first fluid is infused at a flushing rate greater than the first rate as a mixture of first and second fluids are drawn from first and second reservoirs, respectively, into the junction and/or mixing chamber and driven out at the flushing rate. For example, if the first fluid is a non-medicinal fluid (e.g., a saline solution, etc.), it may be desirable to flush the first fluid from the common line at an increased rate in order to infuse the second fluid into the patient as soon as possible. The flushing rate can be equal to the combined first rate plus second rate (as shown) or it can be determined by increasing the combined rate (e.g., the first rate plus the second rate by a flushing factor (e.g., 10%, 20%, 50%, 100%, etc.). The second fluid cannot be infused (e.g., it will not enter the patient) until the internal volume of the common line is cleared of the first fluid. From T3 to T4, the internal volume of the common line has been cleared of the first fluid and beginning at T3 the mixture of the first and second fluids are infused into the patient at a combined rate. From T4 to T5, auto flush is performed: the mixture of the first fluid and the second fluid is infused into the patient at the combined rate as only the first fluid is drawn into the junction and/or mixing chamber and driven out at the combined rate until the internal volume of the common line is cleared of the first and second fluid mixture. The first fluid cannot be infused by itself (e.g., it cannot enter the patient without the second fluid) until the internal volume is cleared of the first and second fluid mixture. After T5, the first fluid is infused at the first rate along the first flow path including the first reservoir after the internal volume of the common line has been cleared of the first and second fluid mixture. In this example, no additional second fluid is infused after T5, although in other embodiments, additional concurrent infusions (of first and second fluid mixtures) and/or secondary infusions (of just the second fluid) may be programmed to occur, as well.

Those skilled in the art will appreciate that the transition between the two infusion modes can be selected as desired for a particular application. In the example of FIG. 4A, a common line auto flush is performed from T4 to T5, but not from T2 to T3. As long as the common line flush volume value is known, the common line auto flush maintaining the first rate between T2 and T3 can be performed as desired.

Referring to FIG. 4B, graph 530 is the fluid volume delivered at the terminal fluid delivery end of the common line for a first fluid versus time and graph 540 is the fluid volume delivered at the terminal fluid delivery end of the common line for a mixture of the first fluid and a second fluid versus time. From T1 to T2, the first fluid is infused at the first rate along a first flow path including the first reservoir and the second fluid is not infused. From T2 to T3, auto flush occurs and the first fluid is infused at the first rate as a mixture of first and second fluids are drawn from first and second reservoirs, respectively, into a junction and/or mixing chamber, and driven out at a combined rate (as discussed above). The first fluid is infused, driven or displaced until the internal volume of the common line has been cleared of the first fluid. After T3, the mixture of the first and second fluids is infused, driven or displaced at the combined rate (as discussed above) after the internal volume of the common line has been cleared of the first fluid. In one embodiment, the common line is cleared of the first fluid when the monitored volume of the mixture of the first and second fluids driven at the first rate between T2 and T3 is equal to or greater than the common line flush volume value. In this example, no additional second fluid is infused after T3, although in other embodiments, additional concurrent infusions (of first and second fluid mixtures) and/or secondary infusions (of just the second fluid) may be programmed to occur, as well.

### Concurrent Delivery With Common Line Auto Flush

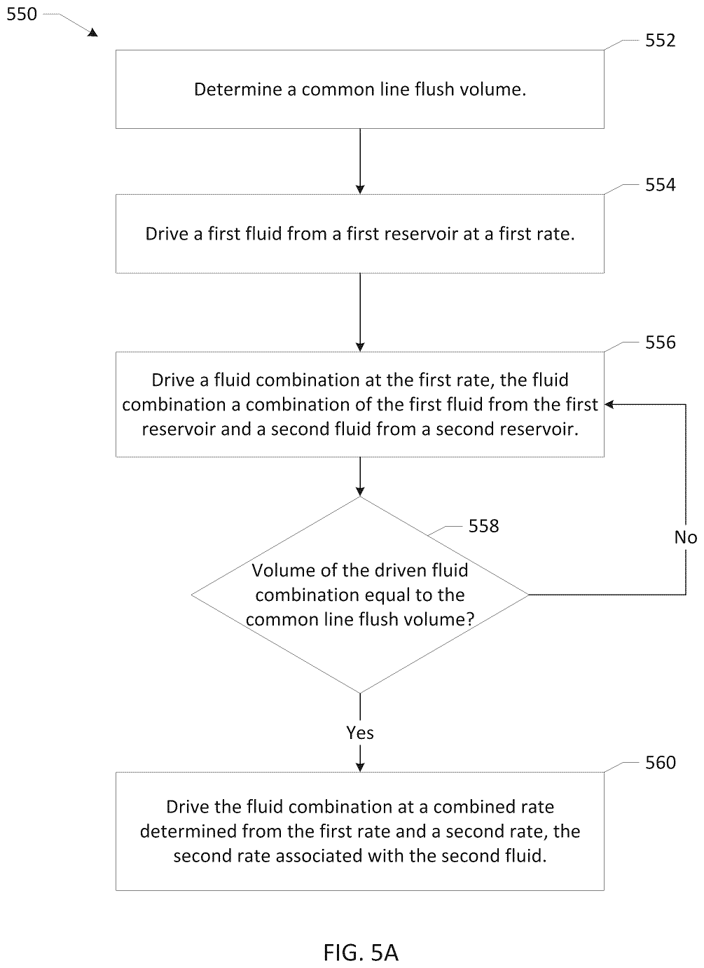

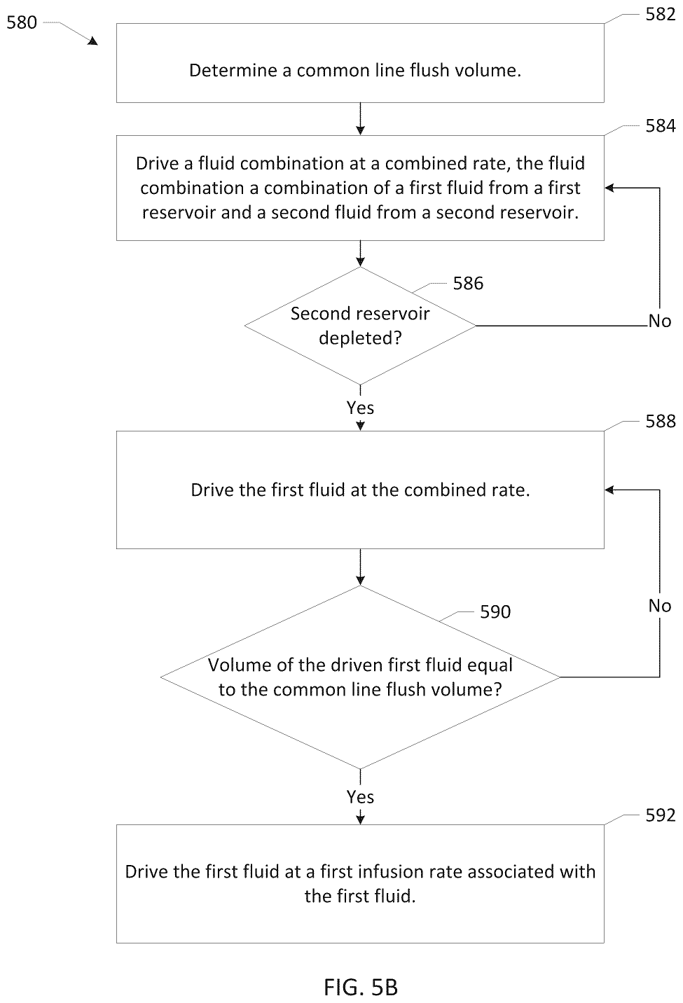

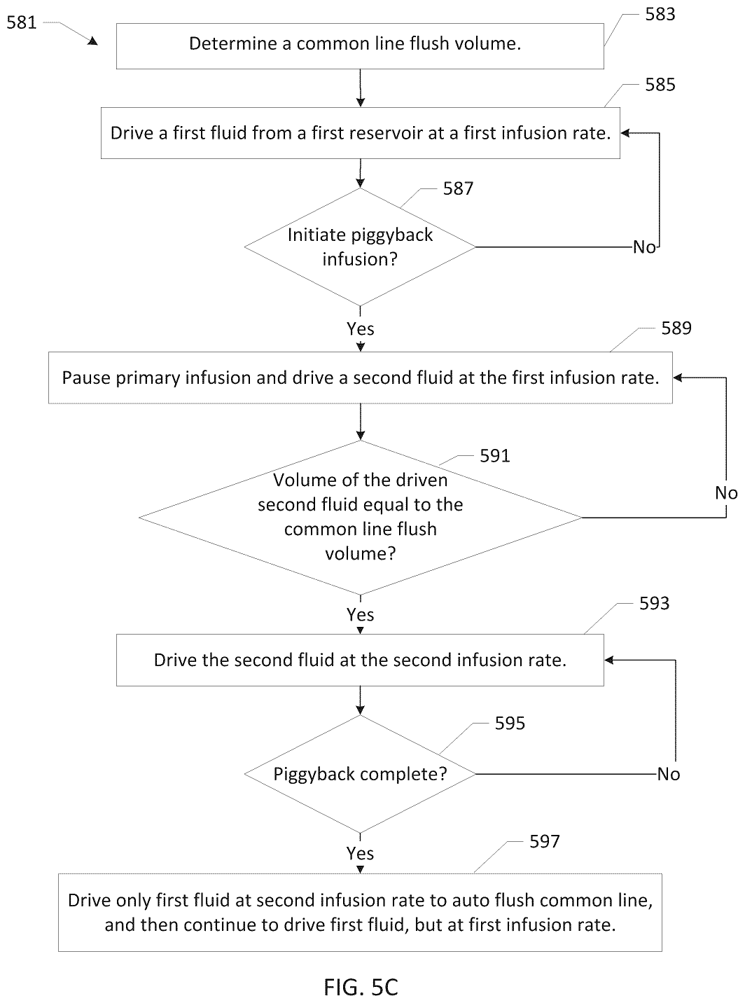

FIG. 5A is a flowchart of an embodiment of a method for concurrent infusion with common line auto flush. The method 550 can be performed with any infusion pump system described herein. In one embodiment, the infusion pump system includes a first reservoir containing a first fluid, a second reservoir containing a second fluid, a junction in fluid communication with the first reservoir and the second reservoir, an optional mixing chamber at or in fluid communication with the junction, and a common line in fluid communication with the junction and/or mixing chamber at one end and having a terminal fluid delivery end, and an infusion pump operable to drive fluid through the common line. The method 550 can be performed by any of the systems discussed herein. In an embodiment, some or all aspects of the method 550 are stored as programmed instructions to be executed by an infusion pump flow controller (e.g., flow controller 235). The method 550 can be used with an infusion pump system and infusion pump as described in FIGS. 1A, 1B, & 2 above. A drug library may include an indication (e.g., flag, value, etc.) to enable or disable concurrent infusion with auto flush, as described with respect to FIG. 5A. In this example, the infusion pump infuses a first fluid on a first flow path at a first rate and switches to a concurrent infusion mode during which it infuses a mixture of the first fluid and a second fluid, maintaining the first rate long enough to clear the remaining first fluid from the common line before changing to a combined rate for infusing the mixture of the first and second fluids.

Referring to FIG. 5A, at block 552, the flow controller 235 determines a common line flush volume value. As discussed above, the common line flush value can be received based on a user input via any of the user interfaces discussed above. In an embodiment, the flow controller 235 can automatically retrieve the common line flush volume value from the memory 233 or over a network (e.g., from a drug library or other database), or by wirelessly reading information from a tag associated with the common line and using the information to retrieve the common line flush volume from the memory or over the network. The common line flush volume may be predetermined for particular fluids. The common line flush volume may also depend on the VTBI or rate of the infusion.

At block 554, a first infusion mode to infuse the first fluid at a first infusion rate begins. The first fluid is infused or driven at a first infusion rate along a first flow path that includes the first reservoir, the junction, the optional mixing chamber, and the common line. The infusion of the first fluid can be controlled by the flow controller 235 based on a control signal to activate the pump or other mechanical system. In some embodiments, the infusion of the first fluid can also be based on a user input or user control of the pump or the mechanical system. During the first infusion mode, the infusion pump drives the first fluid from the first reservoir at the first infusion rate. At block 556, the flow controller 235 can determine to switch from the first infusion mode to a concurrent infusion mode. During an auto flush period, at block 556, the infusion pump drives a mixture or combination of the first fluid and the second fluid toward the common line at the first rate. By driving the combination of the first and second fluids at the first rate, the first fluid remaining in the common line is flushed and delivered to the patient at the same rate as therapeutically required. In some embodiments, during the auto flush period, the infusion pump drives the combination of the first fluid and the second fluid at a combined rate, instead of the first rate. For example, it may be advantageous to use a combined rate to more quickly flush the common line, particularly when the fluid being flushed from the common line is a non-medicinal fluid, such as saline, or other non-medicinal fluid. The combined rate can be determined using any of the methods described herein. For example, the combined rate may be determined as the sum as the first and second rates. The flow controller 235 can use control signals to control the driving of the mixture of the first fluid and the second fluid and to control the rate of delivery. It also may be desirable to flush the common line of a non-medicinal first fluid such as saline, at a rate even higher than the combined rate to expedite delivery of the second medication. In scenarios where drug library-defined limits are assigned for one or both of the two fluid delivery rates, the pump system may allow overrides of the upper rate limit for one or both of the fluids during the common line flush. For example, the pump system could effectively apply these delivery limits upon delivery to the patient, versus upon delivery from the pump. In another embodiment, the method 550 of FIG. 5A may be modified at block 556 such that the infusion pump drives a fluid combination at a rate that is a ratio of a first programmed first fluid rate and a programmed second fluid rate.

At block 558, the flow controller 235 can monitor volume of the mixture of first and second fluids driven at the first rate. The flow controller 235 can determine when the monitored volume is equal to the common line flush volume value. When it is determined that the monitored volume equals or exceeds the common line flush volume, the method 550 proceeds to block 560, where the flow controller 235 continues driving the mixture of the first and second fluids, but at the combined rate. In some embodiments, the flow controller 235 can measure an amount of time before changing the rate of the mixture fluid delivery to the combined rate. In one embodiment, the flow controller 235 can further include incrementing a first fluid displayed volume and a second fluid displayed volume by a proportion of the monitored volume when the monitored volume is equal to or greater than an internal volume of the common line. The proportion of monitored volume to be incremented for each of the first and second fluids can be equal to the proportion of first and second flow rates associated with the first and second fluids, respectively. For example, if the first flow rate is 10 ml/hr and the second flow rate is 5 ml/hr, the proportions of the monitored value incremented on the first and second volume displays will have a 2:1 ratio. If the monitored volume is 3 ml, then the display of the first fluid value will be increased by 2 ml and the display of the second fluid value will be increased by 1 ml. The flow controller 235 can thus accurately track the rate, time, and an amount of each fluid delivered to the patient. In some embodiments, the flow controller 235 executes only some of the steps described above with respect to FIG. 5A. Furthermore, the flow controller 235 can change the order of the steps, include additional steps, or modify some of the steps discussed above.

The common line flush volume value can be selected as desired for a particular application. In one embodiment, the common line flush volume value is an internal volume of the common line. In another embodiment, the common line flush volume value is an internal volume of the common line plus or minus an adjustment volume. The adjustment volume can be any volume desired as a safety factor to assure that the common line is free of the first fluid before the second fluid is infused at the second rate.

In one embodiment, the method 550 further includes incrementing a first fluid displayed volume by the monitored volume when driving a mixture of the first and second fluids at the first rate. The first fluid displayed volume is incremented by the monitored volume when the monitored volume is equal to or greater than an internal volume of the common line.

In some embodiments, the method 550 ends after the concurrent infusion at the combined rate ends. However, in other embodiments, the method 550 continues concurrent delivery of the first and second fluids until one of the fluids is depleted or until the desired volume of one of the fluids has been delivered. In such case, for example, when the second fluid reservoir is depleted, the infusion continues according to the method 580 discussed below with respect to FIG. 5B. If instead, the concurrent infusion continues until the desired volume of one of the fluids has been delivered, then the infusion may continue according to a slightly modified method 580, as discussed below with respect to FIG. 5B.

### Concurrent Delivery to Infusion Completion With Common Line Auto Flush

FIG. 5B illustrates a method 580 of safely performing a concurrent infusion of first and second fluids until the volume of the second fluid reservoir is depleted (e.g., totally depleted or emptied of the second fluid), such that no second fluid or substantially no second fluid remains in the second reservoir. The method 580 can be performed by a flow controller (e.g., flow controller 235) alone and/or in conjunction with the method 550 of FIG. 5A. For example, method 580 may be performed beginning at block 586 and following block 560 of method 550 of FIG. 5A. A drug library may include an indication (e.g., flag, value, etc.) to enable or disable infusion until depletion functionality, as described with respect to FIG. 5B.

At block 582, the method 580 determines a common line flush volume of a common line. Any of the methods described herein may be used to determine the common line flush volume. At block 584, a concurrent infusion occurs, where a fluid combination is driven by an infusion pump at a combined rate. The fluid combination includes a mixture of a first fluid drawn into a junction and/or mixing chamber from a first reservoir and a second fluid drawn into the junction and/or mixing chamber from a second reservoir. As discussed herein, a first infusion rate may be associated with the infusion of the first fluid and a second infusion rate may be associated with the infusion of the second fluid. The ratio of the volumes of first and second fluids drawn into the mixing chamber is equal to the ratio of the ratio of first and second infusion rates. The fluid combination is driven from the junction and/or mixing chamber to the common line at a combined rate, which may be determined according to any of the methods described herein. For example, the combined rate may be determined as the sum of the first and second infusion rates.

At block 586, the method 580 determines whether the second reservoir has been depleted. For example, a sensor can detect whether there is air or air bubbles in the line between the junction and the second reservoir. If the method 580 does not determine that the second reservoir is depleted, the method 580 returns to block 584. If the method 580 determines that the second reservoir has been depleted, the method 580 proceeds to block 588. The method 580 may also optionally cause the infusion pump to at least partially back-prime the line between the junction and the second reservoir. For example, the infusion pump may pump some fluid from the first reservoir to force fluid into the line between the junction and the second reservoir in order to remove air from the line (or at least the portion of the line near the junction).

In a modified version of method 580, at block 586 the method 580 instead determines whether a desired or programmed volume of the second fluid has been delivered. For example, if the infusion pump was programmed to delivery only 100 ml of the second fluid during concurrent delivery mode, the method 580 would determine whether 100 ml of the second fluid had been delivered. In another embodiment, the method 580, determines whether a desired volume of second fluid has been delivered by receiving a command to stop an infusion of the second fluid. When a user provides an input to stop the infusion, the method 580 determines that the desired volume of second fluid has been delivered. If so, the method 580 continues to block 588. If not, the method 580 returns to block 584.

At block 588, the method 580 stops drawing fluid from the second reservoir, and instead only draws fluid from the first reservoir. The method 580 drives the first fluid to the common line at the combined rate in order to auto flush or clear the volume of the common line of the fluid combination remaining in the common line. In the case when there is a drug-library defined limit on the first fluid, the pump system may need to allow an override of this limit in order to support pumping of the first fluid at the combined rate. In other words, drug library-defined delivery limits for the first fluid would apply at the patient, versus at the pump.

At block 590, the method 580 monitors the volume of first fluid driven at the combined rate and determine when the monitored volume equals or exceeds the common line flush volume. If the monitored volume is not equal to the common line flush volume, the method 580 returns to block 588. If the monitored volume is equal to or exceeds the common line flush volume, the method 580 proceeds to block 592.

At block 592, the method 580 continues to draw the first fluid from the first reservoir, but at the first rate. In some embodiments, the method 580 can measure an amount of time before changing the rate of the first fluid delivery to the first rate. In one embodiment, the method 580 can further include incrementing a first fluid displayed volume and a second fluid displayed volume by a proportion of the monitored volume when the monitored volume is equal to or greater than an internal volume of the common line. The proportion of monitored volume to be incremented for each of the first and second fluids can be equal to the proportion of first and second flow rates associated with the first and second fluids, respectively. For example, if the first flow rate is 10 ml/hr and the second flow rate is 5 ml/hr, the proportions of the monitored value incremented on the first and second volume displays will have a 2:1 ratio. If the monitored volume is 3 ml, then the display of the first fluid value will be increased by 2 ml and the display of the second fluid value will be increased by 1 ml. The method 580 can thus accurately track the rate, time, and an amount of each fluid delivered to the patient. In some embodiments, the method 580 executes only some of the steps described above with respect to FIG. 5B. Furthermore, the method 580 can change the order of the steps, include additional steps, or modify some of the steps discussed above

### Sequential Delivery to Reservoir Depletion With Common Line Auto Flush

FIG. 5C illustrates a method 581 of safely performing a sequential infusion (sometimes referred to as a piggyback infusion) of a first fluid at a first infusion rate until the infusion of the first fluid at the first rate is stopped and the infusion switches to an infusion of a second fluid at a second infusion rate. The method 581 can be performed by a flow controller (e.g., flow controller 235) alone and/or in conjunction with the method 550 of FIG. 5A or the method 580 of FIG. 5B. A drug library may include an indication (e.g., flag, value, etc.) to enable or disable infusion until reservoir depletion functionality, as described with respect to FIG. 5B.

At block 583, the method 581 determines a common line flush volume of a common line. Any of the methods described herein may be used to determine the common line flush volume. At block 585, a primary infusion occurs, where a first fluid is driven by an infusion pump at a first infusion rate. The first fluid is drawn into a junction and/or mixing chamber from a first reservoir. The first infusion rate may be associated with the infusion of the first fluid and a second infusion rate may be associated with an infusion of the second fluid. The first fluid is driven from the junction and/or mixing chamber to the common line at the first infusion rate.

At block 587, the method 581 determines whether to pause the first infusion and initiate a “piggyback” infusion, or infusion of a second fluid at a second rate. If the method 581 determines that the second fluid program should be initiated, the method 581 proceeds to block 589. If not, the method 581 returns to block 585.

At block 589, the method 581 stops drawing fluid from the first reservoir (pauses the primary infusion), and instead only draws fluid from the second reservoir. The method 581 drives the second fluid to the common line at the first infusion rate in order to auto flush or clear the volume of the common line of the first fluid remaining in the common line. If the drug library includes limits on the delivery of fluid 2, these limits may need to be allowed to be overridden during the common line flush period defined by block 589. For example, if fluid 1 was programmed at a rate below the lower limit allowed for fluid 2, or if fluid 1 was programmed at a rate above the upper limit allowed for fluid 2, an override of such a limit would be allowed during the common line flush.

At block 591, the method 581 monitors the volume of second fluid driven at the first infusion rate and determines when the monitored volume equals or exceeds the common line flush volume. If the monitored volume is not equal to the common line flush volume, the method 581 returns to block 589. If the monitored volume is equal to or exceeds the common line flush volume, the method 581 proceeds to block 593.

At block 593, the method 581 continues to draw the second fluid from the second reservoir, but at the second infusion rate. In some embodiments, the method 581 can measure an amount of time before changing the rate of the first fluid delivery to the second infusion rate. In one embodiment, the method 581 can further include incrementing a first fluid displayed volume by the monitored volume when the monitored volume is equal to or greater than an internal volume of the common line. The method 581 can thus accurately track the rate, time, and an amount of each fluid delivered to the patient. In some embodiments, the method 581 executes only some of the steps described above with respect to FIG. 5B. Furthermore, the method 581 can change the order of the steps, include additional steps, or modify some of the steps discussed above. In some embodiments, it may be preferable to infuse the second fluid at a rate that exceeds the first infusion rate until the common line (filled with non-medicinal fluid) is cleared, in order to more quickly introduce the second (medicinal) fluid to the patient. If a drug library defined limit for fluid 2 is present, the pump system may permit an override of this limit to allow pumping of fluid 2 at this increased rate. Similarly, there may be limits on fluid 1 delivery rates that should be considered by the pump system, imposing a limit on fluid 2 pumping rates intended to displace common line volume. At block 595, the method 581 determines whether the piggyback infusion is complete. For example, the method 581 may determine that the second reservoir is depleted of fluid, that a desired volume of fluid has been infused, that a desired infusion duration period has been reached, etc. In one embodiment, a sensor determines that air is detected within the fluid line. If the piggyback infusion is not complete, the method 581 returns to block 593. If the piggyback infusion is complete, the method 581 proceeds to block 597. At block 591, the primary infusion, e.g., the infusion of the first fluid, is resumed, though at the second infusion rate until the driven first fluid volume is equal to or greater than the common line volume. In the case where the first fluid has a drug library defined limit(s), the pump system may need to support an override of such a lower or upper limit to support pumping at the rate programmed for the second fluid. Method 581 then continues to drive the first fluid, but now at the first infusion rate.

The method 581 may also optionally cause the infusion pump to at least partially back-prime the line between the junction and the second reservoir after air is recognized at the depletion of the second fluid reservoir. For example, the infusion pump may pump some fluid from the first reservoir to force fluid into the line between the junction and the second reservoir in order to remove air from the line (or at least the portion of the line near the junction).

### Intermittent Concurrent Delivery

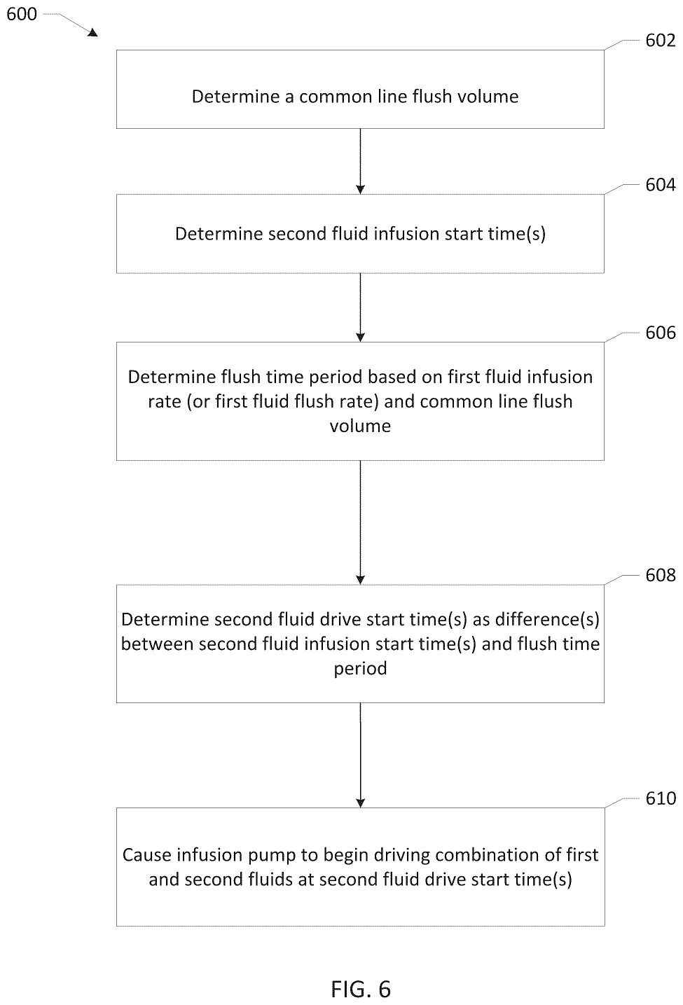

FIG. 6 illustrates a method 600 of scheduling intermittent concurrent deliveries. Method 600 can be performed by an infusion pump, a flow controller (e.g., flow controller 235), and/or alone or in conjunction with the method 550 of FIG. 5A and/or method 580 of FIG. 5B. Method 600 may be performed when it is desired to deliver a secondary infusion (e.g., deliver a second fluid via a concurrent infusion with a first fluid) multiple times per day at specific start times. Method 600 enables an infusion pump to determine a time to start an auto flush procedure to assure that the second fluid is infused into the patient (e.g., enters the patient) at the desired, specific start times.

For example, if a common line volume will take 10 minutes to flush at the primary (first) infusion rate, then the concurrent infusion will initiate an auto flush process (infusing a mixture of first and second fluids at the first infusion rate to flush the first fluid out of the common line tubing) 10 minutes before the desired secondary infusion start time (e.g., 10 minutes before the second fluid is to enter the patient).

At block 602, the method 600 determines a common line flush volume. However, the method may skip block 602 if the common line flush volume has already been determined. At block 604, the method 600 determines one or more second fluid infusion start times. For example, the method 600 may receive or download schedule information corresponding to desired start times to infuse a second fluid into a patient. The schedule information may define specific times during the day (e.g., 8 am, noon, 4 pm, 8 pm, etc.), it may define a number of infusions per day (e.g., 2, 3, 4, 6infusions per day, etc.), or it may define an interval between second fluid infusions (e.g., one bag of second fluid every 4 hours, etc.). The schedule information may be used to determine one or more second fluid infusion start times.

At block 606, the method 600 determines a flush time period based on the first fluid infusion rate (or first fluid flush rate if a faster flush rate is desired for the particular, e.g., non-medicinal, first fluid) and the common line flush volume. For example, the flush time period may be determined by dividing the common line flush volume by the first fluid infusion rate (or first fluid flush rate). The flush time period represents the amount of time it will take to flush remaining fluid from the common line between the junction (or mixing chamber) and the common line distal end when fluid is driven at the first (or first fluid flush) rate.

At block 608, the method 600 determines second fluid drive start times, which correspond to the actual times that the infusion pump will begin to draw first fluid from a first reservoir and second fluid from a second reservoir, and drive the mixture of first and second fluids to the common line at the first (or first fluid flush) rate. In one embodiment, the method 600 may determine the second fluid drive start times by subtracting the flush time period from each of the second fluid infusion start times. For example, if the flush time period is determined to be 20 minutes and the second fluid infusion start times are 8:00 am, 2:00 pm, and 8:00 pm, then the second fluid drive start times may be determined as 7:40 am, 1:40 pm, and 7:40 pm. By initiating an auto flush concurrent infusion at the second fluid drive start times, a mixture of the second fluid and the first fluid will reach the patient and will be infused into the patient (e.g., enter the patient's body) at the second fluid infusion start times. At block 610, the method causes the infusion pump to initiate such auto flush concurrent infusions at the second fluid drive start times.

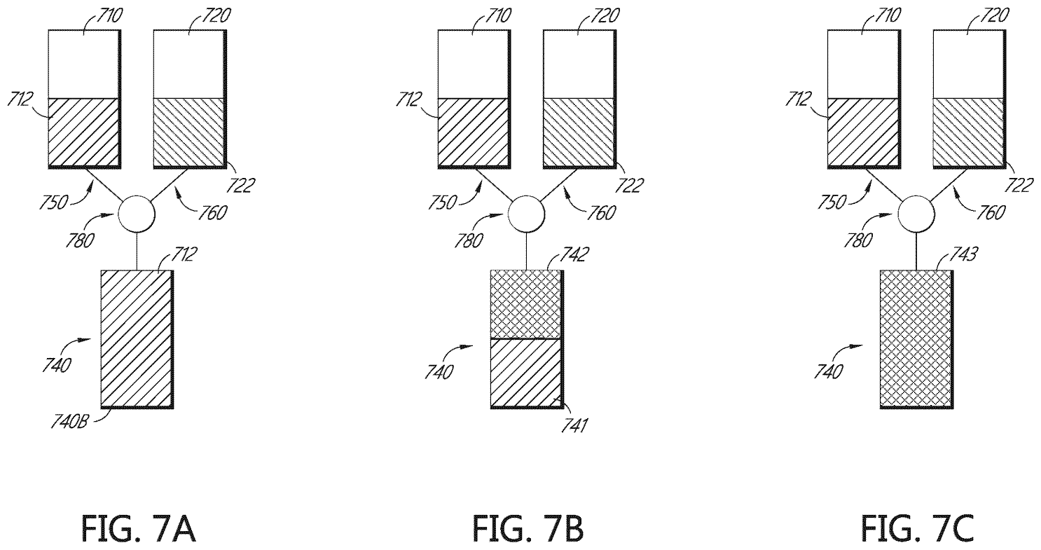

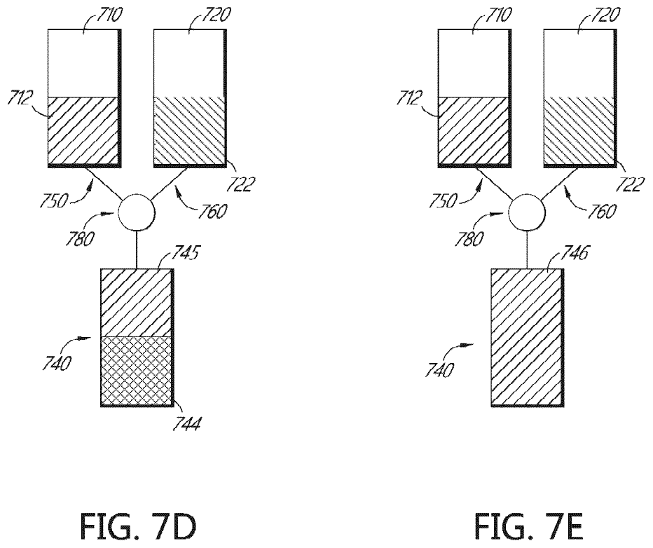

FIGS. 7A-7E are schematic diagrams of use for an infusion pump system with concurrent infusion and common line auto flush in accordance with the present invention. FIGS. 7A-7E illustrate switching from infusing a first fluid to infusing a mixture or combination of first and second fluids, then switching back to infusing the first fluid, while accounting for the previously infused fluid in the common line. In this example, the infusion pump is infusing a first fluid on a first flow path at a first rate and switches to infusing a mixture or combination of first and second fluids on a second flow path, maintaining the first rate long enough to clear the remaining first fluid from the common line before changing to a combined rate for infusing the mixture or combination of first and second fluids. The infusion pump then switches to infusing a first fluid on the first flow path, maintaining the combined rate long enough to clear the remaining mixture or combination of first and second fluids from the common line before changing to a first rate for infusing the first fluid.

Referring to FIG. 7A, the first fluid 712 is delivered to the terminal end 740B of a common line 740 at a first rate along a first flow path 750 including the first reservoir 710, the junction 780, an optional mixing chamber (not shown), and the common line 740. The first fluid 712 is indicated by upward from left to right diagonal lines. Referring to FIG. 7B, the infusion has changed to a concurrent mode. During the concurrent mode, first fluid 712 is drawn from the first reservoir 710 and second fluid 722 is drawn from the second reservoir 720. The second fluid 722 is indicated by downward from left to right diagonal lines. The mixture of first and second fluids 712, 722 is driven by the infusion pump into the common line 740. During this auto flush mode of concurrent delivery, the common line 740 contains first common line fluid 741 remaining from the initial infusion of the first fluid 712 and indicated by the upward diagonal lines, and second common line fluid 742 (the mixture of the first and second fluids 712, 722) indicated by the hashed lines. The flow rate remains at the first rate because the remaining first common line fluid 741 is being delivered to the terminal fluid delivery end 740B or to the patient when connected. Referring to FIG. 7C, none of the first fluid remains in the common line 740, so the second common line fluid 743 (the mixture of the first and second fluids 712, 722) is driven at the combined rate.

The infusion pump system can subsequently switch back to infusing only the first fluid (for example, after a predetermined time period, after a predetermined volume of combined first and second fluids are infused, after a predetermined volume of the second fluid is infused, or after the infusion pump determines that the second reservoir has been depleted of the second fluid, etc.). Referring to FIG. 7D, the infusion mode has changed from concurrent delivery to primary delivery (infusing only first fluid 712 from the first reservoir 710). Initially, the common line 740 still contains a mixture of the first and second fluids 712, 722 (represented by the hashed lines) as second common line fluid 744 remaining from the previous infusion, and first common line fluid 745 (the first fluid 712 alone) indicated by the upward diagonal lines. The flow rate remains at the combined rate because the remaining second common line fluid 744 is being delivered. Referring to FIG. 7E, none of the mixture of first and second fluids remains in the common line 740, so the first common line fluid 746 is driven at the first rate along the first flow path 750.

While the embodiments of the invention disclosed herein are presently considered to be preferred, various changes, rearrangement of steps, and modifications can be made without departing from the scope of the invention. The scope of the invention is indicated in the appended claims, and all changes that come within the meaning and range of equivalents are intended to be embraced therein.

What is claimed is:

1. A method for controlling operation of an infusion pump of an infusion pump system, the infusion pump system comprising a reservoir configured to hold a medicinal fluid, a fluid line in fluid communication with the reservoir and having a terminal fluid delivery end, and the infusion pump, wherein the infusion pump is operable to drive fluid through the fluid line toward the terminal fluid delivery end, the method comprising:
determining a fluid line volume corresponding to a volume of the fluid line, wherein the fluid line comprises a fluid line volume of a non-medicinal fluid;
drawing the medicinal fluid from the reservoir to deliver the medicinal fluid from the reservoir to the terminal fluid delivery end;
infusing the medicinal fluid at a flushing rate into the fluid line, wherein infusing the medicinal fluid at the flushing rate into the fluid line causes displacement of a volume of the non-medicinal fluid remaining in the fluid line and infusion of the non-medicinal fluid out of the terminal fluid delivery end at the flushing rate;
determining that an infused volume of the medicinal fluid equals or exceeds the fluid line volume; and
reducing infusion to an infusion rate lower than the flushing rate.

2. The method of claim 1, further comprising determining the flushing rate based on the non-medicinal fluid.

3. The method of claim 1, further comprising determining the flushing rate as a least one of the infusion rate or a second infusion rate associated with the non-medicinal fluid, increased by a flushing rate factor based on the non-medicinal fluid.

4. The method of claim 1, further comprising receiving the fluid line volume from a user input.

5. The method of claim 1, further comprising retrieving the fluid line volume from a computer-readable memory.

6. The method of claim 1, further comprising obtaining the fluid line volume over a network.

7. The method of claim 1, wherein the fluid line volume is predetermined.

8. The method of claim 1, further comprising determining the fluid line volume based on the non-medicinal fluid.

9. The method of claim 1, further comprising receiving instructions to flush the non-medicinal fluid and to infuse the medicinal fluid via a user interface.

10. The method of claim 1, wherein infusing the medicinal fluid at the flushing rate comprises delivering the medicinal fluid at a rate outside of drug library defined rate limits associated with the medicinal fluid.

11. The method of claim 1, further comprising:
determining that an infusion of the medicinal fluid has completed;
drawing the non-medicinal fluid from a second reservoir without drawing the medicinal fluid from the reservoir;
infusing the non-medicinal fluid at a second rate;
determining that a volume of the non-medicinal fluid infused at the second rate equals or exceeds the fluid line volume; and
changing infusion of the non-medicinal fluid from the infusion rate to a third rate.

12. The method of claim 11, wherein determining that infusion of the medicinal fluid has completed comprises comparing a volume of the medicinal fluid infused to a programmed volume to infuse.

13. The method of claim 11, wherein determining that infusion of the medicinal fluid has completed comprises receiving an instruction to stop infusing the medicinal fluid.

14. The method of claim 11, wherein determining that infusion of the medicinal fluid has completed comprises determining that the reservoir has been depleted of the medicinal fluid.

15. A control system for controlling operation of an infusion pump system, the infusion pump system comprising a reservoir configured to hold a medicinal fluid, a fluid line in fluid communication with the reservoir and having a terminal fluid delivery end, and an infusion pump, wherein the infusion pump is operable to drive fluid through the fluid line toward the terminal fluid delivery end, the control system comprising one or more hardware processors configured to:
determine a fluid line volume corresponding to a volume of the fluid line, wherein the fluid line comprises a fluid line volume of a non-medicinal fluid;
draw the medicinal fluid from the reservoir to deliver the medicinal fluid from the reservoir to the terminal fluid delivery end;
infuse the medicinal fluid at a flushing rate into the fluid line, wherein infusing the medicinal fluid at the flushing rate into the fluid line causes displacement of a volume of the non-medicinal fluid remaining in the fluid line and infusion of the non-medicinal fluid out of the terminal fluid delivery end at the flushing rate;
determine that an infused volume of the medicinal fluid equals or exceeds the fluid line volume; and
reduce infusion to an infusion rate lower than the flushing rate.

16. The control system of claim 15, wherein the fluid line volume is obtained from a user input.

17. The control system of claim 15, wherein the fluid line volume is obtained from a computer-readable memory.

18. The control system of claim 15, wherein medicinal fluid is delivered at a rate outside of drug library defined rate limits associated with the medicinal fluid.

19. The control system of claim 15, wherein the one or more hardware processors are further configured to:
determine that an infusion of the medicinal fluid has completed;
draw the non-medicinal fluid from a second reservoir without drawing the medicinal fluid from the reservoir;
infuse the non-medicinal fluid at a second rate;
determine that a volume of the non-medicinal fluid infused at the second rate equals or exceeds the fluid line volume; and
change infusion of the non-medicinal fluid from the infusion rate to a third rate.

20. The control system of claim 15, wherein the flushing rate is a least one of the infusion rate or a second infusion rate associated with infusion of the non-medicinal fluid, increased by a flushing rate factor based on the non-medicinal fluid.
